(19) United States
(12) Patent Application Publication
(10) Pub. No.: US 2015/0133410 A1
(43) Pub. Date: May 14, 2015

(54) TOPICAL OPHTHALMIC, OTIC, AND NASAL COMPOSITIONS OF CIPROFLOXACIN

(71) Applicants: Nilesh Parikh, Irvine, CA (US); William Hite, Winchester, CA (US)

(72) Inventors: Nilesh Parikh, Irvine, CA (US); William Hite, Winchester, CA (US)

(21) Appl. No.: 14/080,539

(22) Filed: Nov. 14, 2013

(51) Int. Cl. A61K 31/496; A61K 9/00; A61K 47/18; A61K 31/573; A61K 47/02; A61K 47/38; A61K 47/12; A61K 45/06; A61K 47/34

(52) U.S. Cl. CPC A61K 31/496; A61K 45/06; A61K 9/0048; A61K 9/0046; A61K 9/0043; A61K 47/186; A61K 47/34; A61K 47/02; A61K 47/38; A61K 47/12; A61K 31/573
USPC 514/171; 514/253.08

(57) ABSTRACT

Embodiments of the invention provide aqueous pharmaceutical compositions suitable for topical administration to otic tissue, ocular tissue, nasal tissue, or a combination thereof that contain 0.02%-0.40% w/w of a ciprofloxacin, 0.01%-1.0% w/w of a surfactant, 0.05%-2.5% w/w of an acetic acid-acetate salt buffer, an amount of a tonicity agent sufficient to cause the composition to have an osmolality of about 275-325 mOsm, and water. In such embodiments, the compositions have a pH of from 5.0 to 5.3 and at least about 95% of the ciprofloxacin is in solution at room temperature.

### FIELD OF THE INVENTIONS

The present invention relates to aqueous compositions of ciprofloxacin and optionally other drugs (e.g., anti-inflammatory drugs, such as non-steroidal anti-inflammatory drugs and steroidal anti-inflammatory drugs) that are topically administrable to eye, ear, and nasal tissue and are characterized by a reduced irritation potential due to having a less acidic pH and/or a lesser preservative content than previously known topical, aqueous compositions of ciprofloxacin.

### BACKGROUND OF THE INVENTIONS

The topical ophthalmic use of the broad spectrum antibiotic, ciprofloxacin, in treating ophthalmic infections is known; and topical, ophthalmic formulations of ciprofloxacin are commercially available. For instance, CILOXAN is a commercially available ciprofloxacin ophthalmic solution indicated for the topical treatment of corneal ulcers caused by susceptible strains of Pseudomonas aeruginosa, Serratia marcestens, Staphylococcus aureus, Staphylococcus epidermidis, Streptococcus pneumoniae, and Streptococcus (Viridans Group) and conjunctivitis caused by susceptible strains of Haemophilus influenzae, Staphylococcus aureus, Staphylococcus epidermidis, Streptococcus pneumoniae.

The topical otic use of ciprofloxacin in combination with anti-inflammatory steroids such as hydrocortisone or dexamethasone in treating otic infections and attendant inflammation is known; and topical, otic formulations of ciprofloxacin and hydrocortisone or dexamethasone are commercially available. For instance, CIPRO HC is a ciprofloxacin and hydrocortisone otic suspension indicated for the topical treatment of acute otitis externa caused by susceptible strains of Pseudomonas aeruginosa, Staphylococcus aureus, and Proteus mirabilis. CIPRODEX is a commercially available ciprofloxacin and dexamethasone otic suspension indicated for the topical treatment of acute otitis media caused by susceptible isolates of Staphylococcus aureus, Streptococcus pneumoniae, Haemophilus influenzae, Moraxella catarrhalis, and Pseudomonas aeruginosa and acute otitis media caused by Staphylococcus aureus or Pseudomonas aeraginosa.

Topical ophthalmic and otic formulations of ciprofloxacin, alone or in combination with an anti-inflammatory steroid, typically contain preservative(s) and have a pH of approximately 4.5 or less. CIPRODEX and CILOXAN contain the preservative, benzalkonium chloride, and CIPRODEX further contains the preservative, boric acid. CIPRODEX contains an acetic acid-sodium acetate buffer system and is pH 4.5, close to the pKa of the buffer system. CILOXAN also contains an acetic acid-sodium acetate buffer.

WO1990/001933 teaches that ophthalmic products are typically packaged in multidose forms in which preservatives are required to prevent microbial contamination during use. It teaches topical, ophthalmic formulations of ciprofloxacin and dexamethasone, pH 5.5±0.5, that contain benzalkonium chloride as preservative. U.S. Pat. No. 5,843,930 teaches topical, otic formulations of ciprofloxacin and hydrocortisone, pH 4.75. That patent teaches that, in aqueous solutions containing from about 0.2 to about 1 weight percent of ciprofloxacin hydrochloride, crystalline precipitation of ciprofloxacin was observed to occur at pH's above 5.5 at room temperature and at pH's above 5 at 5° C. U.S. Pat/ No. 6,284,804, the content of which is hereby incorporated by reference in its entirety, teaches topical ophthalmic, otic, and nasal formulations of ciprofloxacin and dexamethasone, pH 4.5±0.2, and methods for their preparation, in which the ciprofloxacin is substantially in solution.

The acidic pH of topical, ophthalmic otic, and nasal pharmaceutical compositions have well known irritation potential to sensitive tissues of the eye and ear, its irritation potential heightened in the context of bacterial infection and attendant inflammation.

### SUMMARY OF THE INVENTIONS

Embodiments of the invention provide aqueous pharmaceutical compositions suitable for topical administration to otic tissue, ocular tissue, nasal tissue, or a combination thereof that contain 0.02%-0.40% w/w of a ciprofloxacin base, 0.01%-1.0% w/w of a surfactant, 0.05%-2.5% w/w of an acetic acid-acetate salt buffer, an amount of a tonicity agent sufficient to cause the composition to have an osmolality of about 275-325 mOsm, and water. In such embodiments, a pH of the compositions is from 5.0 to 5.3 and at least about 95% of the ciprofloxacin is in solution at room temperature.

In some embodiments, the surfactant is at least one of a polyoxamer, a deoxycholate, a triton, a polysorbate, a tyloxapol, a sodium lauryl suffate, a polyethoxylated castor oil, a lecithin, a CHAPS, a P-40, and a NP-40.

In some embodiments, the acetate salt is a sodium acetate and the tonicity agent is at least one of a sodium chloride, a sodium sulfate, a mannitol, a sorbitol a maltitol, a xylitol, a sucrose, a maltose, a cellobiose, glucose, a fructose, a galactose, a ribose, and a deoxyribose.

In some embodiments, the compositions further contain 0.01%-2.5% w/w of at least one viscosity building agent selected from the group consisting of a polyvinyl alcohol, a polyvinyl pyrrolidone, a methyl cellulose, a hydroxypropyl methylcellulose, a hydroxyethyl cellulose, a carboxymethyl cellulose, and a hydroxy propyl cellulose

In some embodiments, the compositions further contain 0.005%-1.0% w/w of at least one preservative selected from the group consisting of a boric acid, a benzalkonium chloride, and a benzyl alcohol.

In some embodiments, the compositions further contain 0.005%-0.1% w/w of at least one chelating agent selected from the group consisting of an edetate disodium, an edetate trisodium an edetate tetrasodium; an ethylene glycol tetraacetic acid, and a diethyleneamine pentaacetate.

In some embodiments, the compositions further contain 0.01%-2.5% w/w of a non-steroidal anti-inflammatory drug or an anti-inflammatory steroid drug.

In some embodiments, the compositions further contain 0.01%-0.50% w/w of a dexamethasone or 0.1%-2.50% w/w of a hydrocortisone.

Embodiments of the invention provide aqueous pharmaceutical compositions, suitable for topical administration to otic tissue, ocular tissue, nasal tissue, or a combination thereof, composed of about 0.3% w/w of a ciprofloxacin base, about 0.1% w/w of a dexamethasone alcohol, about 0.53% w/w of a sodium chloride, about 0.2% w/w of a hydroxyethyl cellulose, about 0.05% w/w of a tyloxapol, about 0.03% w/w of a sodium acetate, about 0.04%. w/w of an acetic acid, about 0.01% w/w of a benzalkonium chloride, about 0.01% w/w of an edetate disodium, about 0.6% w/w of a boric acid, and water. In such embodiments, a pH of the compositions is from 5.0 to 5.3 and at least about 95.0% of the ciprofloxacin is in solution at room temperature.

Embodiments of the invention provide aqueous pharmaceutical compositions, suitable for topical administration to otic tissue, ocular tissue nasal tissue, or a combination thereof, composed of about 0.2% w/w of a ciprofloxacin base, about 1.0% w/w of a hydrocortisone, about 0.10% w/w of a polysorbate 20, about 2.0% w/w of a polyvinyl alcohol, about 0.7% w/w of an acetic acid, about 0.41% w/w of a sodium acetate, about 0.41% w/w of a sodium chloride, about 0.15% w/w of a phospholipan 90 H about 0.9% w/w of a sodium chloride, about 0.9% w/w of a benzyl alcohol, and water. Such compositions have a pH between 5.0 and 5.3 and have at least about 95.0% of the ciprofloxacin in solution at room temperature.

Embodiments of the invention provide methods of making a finished, aqueous, pharmaceutical composition that comprises ciprofloxacin at least 95.0% in solution at room temperature. The methods include the steps of: i. making a volume of a surfactant solution that comprises an amount of a surfactant in water, ii. mixing into the surfactant solution an amount of each of a ciprofloxacin base, a tonicity agent, an acetic acid, and an acetate salt to form a first intermediate composition, iii. adding to the first intermediate composition an amount of a base to form either a second intermediate composition or the finished composition, and iv. if the second intermediate composition is formed in step iii., adding to the second intermediate composition a volume of a finishing solution that comprises water.

In such embodiments, the volume of the surfactant solution, the amount of the surfactant, the amount of the ciprofloxacin the amount of the tonicity agent, the amount of the acetic acid, the amount of the acetate salt, the amount of the base, and if the second intermediate composition is formed, the volume of the finishing solution are together operative to set a pH of the finished composition from 5.0 to 5.3, an osmolality of the finished composition between 275 and 326 mOsm, a ciprofloxacin content of the finished composition from 0.02% to 0.40% w/w, a surfactant content of the finished composition from 0.01% to 1.0% w/w, an acetic acid content of the finished composition from 0.05% to 1.25% w/w and an acetate salt content of the finished composition from 0.05% to 1.25% w/w.

In some embodiments, the surfactant is at least one of a polyoxamer, a deoxycholate, a triton, a polysorbate, a tyloxapol, a sodium lauryl sulfate, a polyethoxylated castor oil, a lecithin, a CHAPS, a P-40, and a NP-40.

In some embodiments, the acetate salt is a sodium acetate and the tonicity agent is at least one of a sodium chloride, a sodium sulfate, a mannitol, a sorbitol, a maltitol, a xylitol, a sucrose, a maltose, a cellobiose, a glucose, a fructose, a galactose, a ribose, and a deoxyribose.

In some embodiments, the base is at least one of a sodium hydroxide and a potassium hydroxide.

In some embodiments, the methods further involve mixing into at least one of the surfactant solution, the first intermediate composition, and the second intermediate composition an amount of at least one viscosity building agent selected from the group consisting of a polyvinyl alcohol, a polyvinyl pyrrolidone, a methyl cellulose, a hydroxypropyl methylcellulose, a hydroxyethyl cellulose, a carboxymethyl cellulose, and a hydroxy propyl cellulose. In such embodiments, the amount of the viscosity agent is operative to set a viscosity agent content of the finished composition from 0.01% to 2.5% w/w.

In some embodiments, the methods further involve mixing into at least one of the surfactant solution, the first intermediate composition, and the second intermediate composition an amount of at least one preservative selected from the group consisting of a boric acid, a benzalkonium chloride, and a benzyl alcohol. In such embodiments, the amount of the preservative is operative to set a preservative content of the finished composition from 0.005% to 1.0% w/w.

In some embodiments, the methods further involve mixing into at least one of the surfactant solution, the first intermediate composition, and the second intermediate composition an amount of at least one chelating agent selected from the group consisting of an edetate disodium an edetate trisodium, an edetate tetrasodium; an ethylene glycol tetraacetic acid, and a diethyleneamine pentaacetate. In such embodiments, the amount of the chelating agent is operative to set a chelating agent content of the finished composition from 0.005% to 0.1% w/w.

In some embodiments, the methods further involve mixing into at least one of the surfactant solution, the first intermediate composition, and the second intermediate composition an amount of a non-steroidal anti-inflammatory drug or an anti-inflammatory steroid drug. In such embodiments, the amount of the non-steroidal anti-inflammatory drug or the anti-inflammatory steroid drug is operative to set a non-steroidal anti-inflammatory drug content or an anti-inflammatory steroid drug content of the finished composition from 01% to 2.50% w/w.

In some embodiments, the methods further involve mixing into at least one of the surfactant solution, the first intermediate composition, and the second intermediate composition an amount of an anti-inflammatory drug selected from the group consisting of a dexamethasone and a hydrocortisone. In such embodiments, the amount of the anti-inflammatory drug is operative to set an anti-inflammatory drug content of the finished composition from 0.1% to 2.50% w/w.

### DETAILED DESCRIPTION OF THE INVENTIONS

Ciprofloxacin solutions of increased alkalinity. The prior art teaches that in aqueous compositions of ciprofloxacin suitable for topical application to otic, ocular, and/or nasal tissue, ciprofloxacin is in solution at pH 4.75 and below. U.S. Pat. No, 5,843,930 teaches that, in aqueous preparations of 0.2%-1% w/w ciprofloxacin, the ciprofloxacin undergoes crystalline precipitation at pH above 5.5 at room temperature and at pH above 5 at 5° C. U.S. Pat. No. 6,284,804 teaches methods of preparing topical ophthalmic, otic and nasal formulations of ciprofloxacin and dexamethasone, pH 4.5±0.2, in which the ciprofloxacin is substantially in solution at room temperature. It is presently disclosed that ciprofloxacin formulations made according to the methods described by U.S. Pat. No. 6,284,804, except for being pH 5.0 or 5.5, have amounts of ciprofloxacin in solution, at 25° C., that are significantly reduced compared to the corresponding pH 4.5 formulation. The present invention provides aqueous compositions of ciprofloxacin that are suitable for topical administration to otic, ocular, and/or nasal tissue, have a pH of from 5.0 to 5.3, and surprisingly contain ciprofloxacin substantially in solution at 25° C.

### EXAMPLE 1

Two aqueous ciprofloxacin compositions having the formulations set forth in Table 1 were made by the process set forth in U.S. Pat. No. 6,284,804, except for being pH 5.0 or 5.5.

TABLE 1

| Ingredient | 1 | 2 |
|---|---|---|
| Dexamethasone | 1.0 mg/ml | 1.0 mg/ml |
| Ciprofloxacin base | 3.0 mg/ml | 3.0 mg/ml |
| Benzalkonium chloride (50%) | 0.1 mg/ml | 0.1 mg/ml |
| Tyloxapol | 0.5 mg/ml | 0.5 mg/ml |
| Sodium chloride | 5.3 mg/ml | 5.3 mg/ml |
| Acetic acid | 0.4 mg/ml | 0.4 mg/ml |
| Edetate disodium | 0.1 mg/ml | 0.1 mg/ml |
| Hydroxyethylcellulose | 2.0 mg/ml | 2.0 mg/ml |
| Boric acid | 6.0 mg/ml | 6.0 mg/ml |
| Sodium acetate | 0.3 mg/ml | 0.3 mg/ml |
| Sodium hydroxide | q.s. to pH 5.0 | q.s. to pH 5.5 |
| Purified water | q.s. | q.s. |

The amounts of ciprofloxacin in solution at room temperature were experimentally determined for Formulations 1 and 2 by the HPLC assay described in Example 3 and are reported in Table 2.

TABLE 2

| Formulation | Ciprofloxacin (%) | | pH |
|---|---|---|---|
| 1 | Supernatant | 91.2 | 5.0 |
| | Residue | 5.7 | |
| 2 | Supernatant | 88.3 | 5.5 |
| | Residue | 6.9 | |

### EXAMPLE 2

Six aqueous ciprofloxacin compositions having the formulations set forth in Table 3 were made by the process of the present invention set forth in Example 2.

TABLE 3

| Ingredient | A | B | C | D | E | F |
|---|---|---|---|---|---|---|
| Dexamethasone | 1.0 mg/ml | 1.0 mg/ml | 1.0 mg/ml | 1.0 mg/ml | 1.0 mg/ml | 1.0 mg/ml |
| Ciprofloxacin | 3.0 mg/ml | 3.0 mg/ml | 3.0 mg/ml | 3.0 mg/ml | 3.0 mg/ml | 3.0 mg/ml |
| base | | | | | | |
| Benzalkonium | 0.1 mg/ml | 0.1 mg/ml | 0.1 mg/ml | 0.1 mg/ml | 0.1 mg/ml | 0.1 mg/ml |
| chloride (50%) | | | | | | |
| Tyloxapol | 0.5 mg/ml | 0.5 mg/ml | 0.5 mg/ml | 0.5 mg/ml | 0.5 mg/ml | 0.5 mg/ml |
| Sodium chloride | 5.3 mg/ml | 5.3 mg/ml | 5.3 mg/ml | 5.3 mg/ml | 5.3 mg/ml | 5.3 mg/ml |
| Acetic acid | 0.4 mg/ml | 0.4 mg/ml | 0.4 mg/ml | 0.4 mg/ml | 0.4 mg/ml | 0.4 mg/ml |
| Edetate disodium | 0.1 mg/ml | 0.1 mg/ml | 0.1 mg/ml | 0.1 mg/ml | 0.1 mg/ml | 0.1 mg/ml |
| Hydroxyethylcellulose | 2.0 mg/ml | 2.0 mg/ml | 2.0 mg/ml | 2.0 mg/ml | 2.0 mg/ml | 2.0 mg/ml |
| Boric acid | 6.0 mg/ml | 6.0 mg/ml | 6.0 mg/ml | 6.0 mg/ml | 6.0 mg/ml | 6.0 mg/ml |
| Sodium acetate | 0.3 mg/ml | 0.3 mg/ml | 0.3 mg/ml | 0.3 mg/ml | 0.3 mg/ml | 0.3 mg/ml |
| Sodium hydroxide | q.s. to pH | q.s. to pH | q.s. to pH | q.s. to pH | q.s. to pH | q.s. to pH |
| | 5. | 5.1 | 5.2 | 5.3 | 5.4 | 5.5 |
| Purified water | q.s. | q.s. | q.s. | q.s. | q.s. | q.s. |

Process for Preparation of Ciprofloxacin Solutions of Increased Alkalinity

### Part I—Dexamethasone

Step 1. 600 g of purified water was weighed and transferred to a clean, dry 1000 ml beaker. A magnetic rod was added to the water, and the beaker was placed on a magnetic stirrer. 4.0 g hydroxyethyl cellulose to was added to the stirred, purified water over a period of 15 minutes, followed by continued stirring for 15 minutes.

Step 2. 2.0 g of dexamethasone was added, under continued stirring, to the resultant solution of step 1followed by continued stirring for 10 minutes.

Step 3. The resultant solution of step 2 was autoclaved at 121° C. for 20 minutes, at 15 psi.

Step 4. The autoclaved solution of Step 3 was cooled to 20° C.-25° C. in an ice-bath.

Part II—Ciprofloxacin

Step 5. 1000 g of purified water was weighed and transferred to a clean, dry 2000 beaker. A magnetic rod was added to the water, and the beaker was placed on a magnetic stirrer.

Step 6. 1.0 g of tyloxapol was weighed in a 50 ml beaker. 20 ml of the purified water from step 5 was added to the 1.0 g of tyloxapol and mixed or 10 minutes, then stirred with a spatula until a clear solution was obtained. The dear tyloxapol solution was added back to the remaining purified water of step and stirred for 20 minutes.

Step 7. 7.0 g of ciprofloxacin hydrochloride monohydrate was weighed and added, with stirring, to the resultant solution of step 6, and further stirred for 15 minutes until a dear solution obtained.

Stop 8. 20 ml of the resultant solution from stop 7 was added to 0.4 g of a 50% w/w benzalkonium chloride solution in a 50 ml beaker, mixed with a spatula, then added back to remaining resultant solution of step 7 and stirred for 15 minutes until a clear solution was obtained.

Step 9. 0.6 g of sodium acetate was added, with stirring, to the resultant solution of step 8, and further stirred for 10 minutes until a clear solution was obtained.

Step 10. To 20 ml of the resultant solution of step 9 in a 50 ml beaker, 0.8 g of acetic acid was added, mixed with a spatula for 5 minutes, and then added back to the remaining resultant solution of step 9, followed by stirring until a clear solution obtained.

Step 11. 10.6 g of sodium chloride was weighed and added with stirring, to the resultant solution of step 10, and further stirred for 10 minutes until a clear solution obtained.

Step 12. 12.0 g of boric acid was added, with stirring, to the resultant solution of step 11, and further stirred for 15 minutes until a clear solution obtained.

Step 13. 0.2 g of EDTA was added, with stirring, to the resultant solution of step 12, and further stirred for 1.0 minutes until a clear solution obtained.

Part III—Final Composition

Step 14. The resultant solution of Part I (Step 4) was added to the resultant solution of Part II (Step 13), under homogenization at 3600 RPM.

Step 15. The volume of the resultant solution of Step 14 was brought to 1700 ml with purified water.

Step 16. The pH of the resultant solution of Step 14 was adjusted to pH 5.0 (Formulation A), pH 5.10 (Formulation B) pH 5.20 (Formulation C), pH 5.30 (Formulation D), pH 5.40 (Formulation E), or pH 5.50 (Formulation F), with stirring and adding required amounts of 2 N Sodium Hydroxide solution.

Step 17. The volume of the resultant solution of Step 16 was brought to 2000 ml with purified water.

Step 18. The resultant solution of Step 17 was homogenized at 24000 RPM for 10 minutes.

The amounts of ciprofloxacin in solution and out of solution in Formulations A-F were experimentally determined at room temperature by the high performance liquid chromatography (HPLC) assay described in Example 3 and are reported in Table 4.

TABLE 4

| Formulation | Ciprofloxacin (%) | | pH |
|---|---|---|---|
| A | Supernatant | 100.3 | 5.00 |
| | Residue | 0.6 | |
| B | Supernatant | 98.6 | 5.10 |
| | Residue | 0.8 | |
| C | Supernatant | 98.9 | 5.20 |
| | Residue | 1.3 | |
| D | Supernatant | 94.5 | 5.30 |
| | Residue | 1.7 | |
| E | Supernatant | 89.9 | 5.40 |
| | Residue | 2.1 | |
| F | Supernatant | 91.5 | 5.50 |
| | Residue | 2.8 | |

### EXAMPLE 3

Ciprofloxacin HPLC Assay

A 10.0 g sample was centrifuged at 3500 rpm for 30 minutes. The supernatant was decanted into a separate test tube. The residue and the supernatant (contains ciprofloxacin in solution) were prepared for HPLC determination of ciprofloxacin content.

Residue HPLC Sample Preparation

The residue was dried at 105° C. for 2 hrs and then transferred to a 200 ml volumetric flask. 100 ml of diluent solution was added to the dried residue, and the resultant solution sonicated until the residue was dissolved. Additional diluent solution was added to bring the total volume to 200 ml, 5 ml of which was transferred to a 20 ml volumetric flask and brought to a total volume of 20 ml with diluent solution.

Supernatant HPLC Sample Preparation

2.5 g of supernatant was transferred to a 200 ml volumetric flask, to which 100 ml of diluent solution was added. The resultant solution was sonicated for 5 minutes and then cooled to room temperature. The total volume of the cooled solution was brought to 200 ml with diluent solution.

The diluent solution used to prepare the residue and supernatant HPLC samples was a solution composed of 70% v/v Mobile Phase A and 30% v/v Mobile Phase B. Mobile Phase A was prepared by dissolving 2.72 g of potassium dihydrogen phosphate in 1000 ml water, adding 2 ml of triethylamine, mixing, and adjusting the pH to 3.00 (±0.05) with dilute ortho phosphoric acid solution. The resultant solution was filtered through a 0.45 μm nylon membrane filter. Mobil Phase B was acetonitrile.

HPLC Conditions

10 μl of sample prepared as described above was injected onto a 250 mm×4.6 mm, 5 μm HPLC column packed with a cyanopropyl group silica gel and subjected to the gradient profile set forth in Table 2.

TABLE 2

| Time | % Mobile Phase A | % Mobile Phase B |
|---|---|---|
| 0.00 | 95.0 | 5.0 |
| 6.00 | 70.0 | 30.0 |
| 13.00 | 58.0 | 42.0 |
| 16.00 | 40.0 | 60.0 |
| 18.00 | 95.0 | 5.0 |
| 23.00 | 95.0 | 5.0 |

The run time was 23 min, the flow rate was 1.2 ml/min, the column temperature was 30° C., the sample cooler temperature was room temperature, and the detector was at 254 nm. The ciprofloxacin retention time was 6.5 to 8.5 minutes.

Ciprofloxacin Calculations

The ciprofloxacin content of the residue, supernatant, and standard samples were calculated with Formula 1.

$$Ciprofloxacin mg / ml = \frac{Aspl}{Astd} \times \frac{Wstd ( mg )}{50 ml} \times \frac{5 ml}{100 ml} \times \frac{200 ml}{Wspl ( mg )} \times \frac{P ( \% )}{100} \times \frac{331.34}{367.81} \times Wt \text{/} ml \qquad Formula 1$$

Aspl=average area response of ciprofloxacin obtained in the sample solution
Astd=average area response of five replicate injections for ciprofloxacin obtained in standard solution-I
Wstd=weight of the ciprofloxacin hydrochloride working standard taken in mg.
Wspl=weight of sample in mg
P=purity of ciprofloxacin hydrochloride working standard on as is basis in percentage.
331.34=molecular weight of ciprofloxacin
wt/ml=weight per ml in mg/ ml

As can be seen, Formulations A, B, C, and D have at least 95% ciprofloxacin in solution, surprisingly improved in comparison to Formulations 1 and 2.

The content of U.S. Pat. No. 6,284,804 is hereby incorporated by reference in its entirety.

Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. An aqueous pharmaceutical composition suitable for topical administration to otic tissue, ocular tissue, nasal tissue, or a combination thereof, the composition comprising: 0.02%-0.40% w/w of a ciprofloxacin base, 0.01% -1.0% w/w of a surfactant, 0.05%-2.5% w/w of an acetic acid acetate salt buffer, an amount of a tonicity agent sufficient to cause the composition to have an osmolality of about 275-325 mOsm, and water, wherein a pH of the composition is from 5.0 to 5.3, and wherein at least about 95% of the ciprofloxacin is in solution at room temperature.

2. The composition of claim 1, wherein the surfactant is at least one member selected from the group consisting of a polyoxamer, a deoxycholate, a triton, a polysorbate, a tyloxapol, a sodium lauryl sulfate, a polyethoxylated castor oil, a lecithin, a CHAPS, a P-40, and a NP-40.

3. The composition of claim 2, wherein the acetate salt is a sodium acetate, and wherein the tonicity agent is at least one member selected from the group consisting of a sodium chloride, a sodium sulfate, a mannitol, a sorbitol, a maltitol, a xylitol, a sucrose, a maltose, a cellobiose, a glucose, a fructose, a galactose, a ribose, and a deoxyribose.

4. The composition of claim 3, further comprising 0.01%-2.5% w/w of at least one viscosity building agent selected from the group consisting of a polyvinyl alcohol, a polyvinyl pyrrolidone, a methyl cellulose, a hydroxypropyl methylcellulose, a hydroxyethyl cellulose, a carboxymethyl cellulose, and a hydroxy propyl cellulose

5. The composition of claim 3, further comprising 0.005%-1.0% w/w of at least one preservative selected from the group consisting of a boric acid, a benzalkonium chloride, and a benzyl alcohol.

6. The composition of claim 3, further comprising 0.005% 0.1% w/w at least one chelating agent selected from the group consisting of an edetate disodium, an edetate trisodium, edetate tetrasodium; an ethylene glycol tetraacetic acid, and a diethyleneamine pentaacetate.

7. An aqueous pharmaceutical composition suitable for topical administration to otic tissue, ocular tissue, or both, the composition comprising:
0.02%-0.40% w/w of a ciprofloxacin,
0.1%-2.50% w/w of an anti-inflammatory drug,
0.01%-1.0% w/w of a surfactant,
0.05%-2.5% w/w of an acetic acid acetate salt buffer,
0.01%-2.5% w/w of at least one viscosity building agent,
a tonicity agent in an amount sufficient to bring an osmolality of the composition to 275-325 mOsm, and water, wherein:
a pH of the composition is from 5.0 to 5.3, and wherein at least about 95.0% of the ciprofloxacin is in solution at room temperature.

8. The composition of claim 9, further comprising:
0.005%-0.1% w/w of a preservative, and
0.005%-0.1% w/w of a chelating agent,
wherein:
the anti-inflammatory drug is at least one member of the group consisting of a nonsteroidal anti-inflammatory drug and a steroid drug,
the surfactant is at least one member selected from the group consisting of a polyoxamer, a deoxycholate, a triton, a polysorbate, a tyloxapol, a sodium lauryl sulfate, a polyethoxylated castor oil, a lecithin, a CHAPS, a P-40, and a NP-40,
the acetate salt is a sodium acetate,
one viscosity building agent selected from the group consisting of a polyvinyl alcohol, a polyvinyl pyrrolidone, a methyl cellulose, a hydroxypropyl methylcellulose, a hydroxyethyl cellulose, a carboxymethyl cellulose, and a hydroxy propyl cellulose,
the tonicity agent is at least one member selected from the group consisting of a sodium chloride, a sodium sulfate, a mannitol, a sorbitol, a maltitol, a xylitol, a sucrose, a maltose, a cellobiose, a glucose, a fructose, a galactose, a ribose, and a deoxyribose

9. The composition of claim 8, wherein the composition comprises:
about 0.3% w/w of a ciprofloxacin base,
about 0.1% w/w of a dexamethasone,
about 0.53% w/w of a sodium chloride,
about 0.2% w/w of a hydroxyethyl cellulose,
about 0.05% w/w of a tyloxapol,
about 0.03% w/w of a sodium acetate,
about 0.04% w/w of an acetic acid,
about 0.01% w/w of a benzalkonium chloride,
about 0.01% w/w of an edetate disodium, and
about 0.6% w/w of a boric acid.

10. A method of making a finished, aqueous, pharmaceutical composition that comprises ciprofloxacin at least 95.05 in solution at room temperature, the method comprising the steps of:
i. making a volume of a surfactant solution that comprises an amount of a surfactant in water,
ii. mixing into the surfactant solution an amount of each of a ciprofloxacin base, a tonicity agent, an acetic acid, and an acetate salt to form a first intermediate composition,
iii. adding to the first intermediate composition an amount of a base to form either a second intermediate composition or the finished composition, and
iv. if the second intermediate composition is formed in step iii., adding to the second intermediate composition a volume of a finishing solution that comprises water,
wherein the volume of the surfactant solution, the amount of the surfactant, the amount of the ciprofloxacin, the amount of the tonicity agent, the amount of the acetic acid, the amount of the acetate salt, the amount of the base, and, if the second intermediate composition is formed, the volume of the finishing solution are together operative to set:
a pH of the finished composition from 5.0 to 5.3,
an osmolality of the finished composition between 275 and 325 mOsm,
a ciprofloxacin content of the finished composition from 0.02% to 0.40% w/w,
a surfactant content of the finished composition from 0.01% to 1.0% w/w,
an acetic acid content of the finished composition from 0.05% to 1.25% w/w, and
an acetate salt content of the finished composition from 0.05% to 1.25% w/w.

11. The method of claim 10, wherein the surfactant is at least one member selected from the group consisting of a polyoxamer, a deoxycholate, a triton, a polysorbate, a tyloxapol, a sodium lauryl sulfate, a polyethoxylated castor oil, a lecithin, a CHAPS, a P-40, and a NP-40.

12. The method of claim 11, wherein the acetate salt is a sodium acetate, and wherein the tonicity agent is at least one member selected from the group consisting of a sodium chloride, a sodium sulfate, a mannitol, a sorbitol, a malitol, a xylitol, a sucrose, a maltose, a cellobiose, a glucose, a fructose, a galactose, a ribose, and a deoxyribose.

13. The method of claim 12, wherein the base is at least one member selected from the group consisting of a sodium hydroxide and a potassium hydroxide.

14. The method of claim 12, further comprising mixing into at least one of the surfactant solution, the first intermediate composition, and the second intermediate composition an amount of at least one viscosity building agent selected from the group consisting of a polyvinyl alcohol, a polyvinyl pyrrolidone, a methyl cellulose, a hydroxypropyl methylcellulose, a hydroxyethyl cellulose, a carboxymethyl cellulose, and a hydroxy propyl cellulose, and wherein the amount of the at least one viscosity agent is operative to set a viscosity agent content of the finished composition from 0.01% to 2.5% w/w.

15. The method of claim 12, further comprising mixing into at least one of the surfactant solution, the first intermediate composition, and the second intermediate composition an amount of at least one preservative selected from the group consisting of a boric acid, a benzalkonium chloride, and a benzyl alcohol, and wherein the amount of the at least one preservative is operative to set a preservative content of the finished composition from 0.005% to 1.0% w/w.

16. The method of claim 12, further comprising mixing into at least one of the surfactant solution, the first intermediate composition, and the second intermediate composition an amount of at least one chelating agent selected from the group consisting of an edetate disodium, an edetate trisodium, edetate tetrasodium; an ethylene glycol tetraacetic acid, and a diethyleneamine pentaacetate, and wherein the amount of the at least one chelating agent is operative to set a chelating agent content of the finished composition from 0.005% to 0.1% w/w.

17. The method of claim 12, further comprising mixing into at least one of the surfactant solution, the first intermediate composition, and the second intermediate composition an amount of a non-steroidal anti-inflammatory drug or an anti-inflammatory steroid drug, and wherein the amount of the non-steroidal anti-inflammatory drug or the anti-inflammatory steroid drug is operative to set a non-steroidal anti-inflammatory drug content or a anti-inflammatory steroid drug content of the finished composition from 0.1% to 2.50% w/w.

18. The method of claim 12, further comprising mixing into at least one of the surfactant solution, the first intermediate composition, and the second intermediate composition an amount of an anti-inflammatory drug selected from the group consisting of a dexamethasone and a hydrocortisone, and wherein the amount of the anti-inflammatory drug is operative to set the anti-inflammatory drug content of the finished composition from 0.1% to 2.50% w/w.
